(19) United States
(12) Patent Application Publication
(10) Pub. No.: US 2012/0259240 A1
(43) Pub. Date: Oct. 11, 2012

(54) Method and System for Assessing and Measuring Emotional Intensity to a Stimulus

(75) Inventors: Tim Llewellynn, Saint-Prex (CH); Matteo Sorci, Chavannes-pres-Renens (CH)

(73) Assignee: Nviso Sarl, Lausanne (CH)

(21) Appl. No.: 13/082,758

(22) Filed: Apr. 8, 2011

(51) Int. Cl. A61B 5/00

(52) U.S. Cl. 600/558

(57) ABSTRACT

It is discloses a method and system for measuring and assessing the impact of non-verbal responses of a respondent to a stimulus, the method comprises the steps of presenting a reference stimulus to the respondent; recording immediate non-verbal responses via an imaging device to said presented reference stimulus; presenting a stimulus under test to the respondent; recording immediate non-verbal responses via an imaging device to said presented stimulus under test; presenting a questionnaire with questions on the stimulus under test to the respondent; obtaining verbal responses to the questions; immediately transmitting the recorded image of said non-verbal responses to the reference stimulus and the stimulus under test across a communications network to an image processing unit and after having received said images at said image processing unit automatically calculating emotion probabilities of the non-verbal responses of the respondent from said images; and calculating a emotional intensity score derived from the emotional probabilities. The method described in this invention bridges the gap between verbal self-report and autonomic non-verbal emotional response measurement methods while adding an objective and scientific analyze of non-verbal response to a stimulus.

### FIELD OF THE INVENTION

The present invention concerns methods and a system related to the measurement and assessment of consumer's non-verbal response to a marketing stimulus according to the independent claims.

### BACKGROUND OF THE INVENTION

In the United States, and elsewhere throughout the world, advertising is extensively used to promote consumer and commercial products. The intent of advertising is to leave embedded impressions of brands and products, creating brand awareness and influencing decision-making. It is almost universally accepted that, as between or among commodity products, which are generally similar to one another in content, price, or quality, successful advertising can help a particular product achieve much greater market penetration and financial success than an otherwise similar product.

Advertising and particularly consumer advertising, although a multi-billion dollar industry in the United States alone is an area wherein workers find it extremely difficult to create and reproduce what prove to be consistently successful advertising campaigns. While it is often easy to predict that the response to a particular proposed advertisement or campaign will be unfavorable, it is not known how to assure success on a consistent basis. Accordingly, it is common to find that long after decisions are made and expenditures incurred, that such efforts have simply not been successful, in that the advertisement or campaign failed to produce sales in amounts proportionate to the expenditure of effort and money.

Key to improving this situation lies in understanding the drivers of consumer behavior and unlocking the buyer decision making process, which today, is among the biggest challenges in marketing research. Recent findings in cognitive neuroscience and Neuroeconomics (Loewenstein 2000; Mellers and McGraw 2001) have made it clear that emotions play an even larger role in decision making than so far assumed. The idea of rational decision making and emotion and feelings as noise has ultimately been rejected. Decision-making without the influence of emotions is not possible. Sound and rational decision-making depends on prior accurate emotion processing (Bachara and Damasio, 2005) Thus the importance of including emotional aspects in consumer research is even greater than was earlier recognized.

Neuroscience findings support the notion that emotions can appear prior to cognition but also shows that the influence goes both ways. Neuroscience has given foundation for new research on emotions in consumer research, also known as Neuroeconomics or consumer neuroscience. In advertising neuroscience methods have been applied by e.g. Ambler, Ioannides and Rose (2000). Yoon et al. (2006) test the notion of brand personality, and Erk et al. (2002) made an interesting study of consumer choice between products in form of different car types finding differences in activation of reward areas related to different types of cars.

Despite these latest advancements in our understanding of emotions on consumer decision making, few companies have come close to exploiting emotions in the design of new products, marketing material or advertising campaigns. Perhaps the root of this misplacement can be attributed to the consumer model borrowed from neoclassical economics. From a business perspective, dealing with a rational consumer paradigm is easier. It can be quantified, segmented, and put into a spreadsheet. If emotions can not be measured and analyzed in a comparable way, they can not be managed. Thus there is clear need for new scientific methods for measuring and assessing the impact of emotions on consumers to marketing stimuli which are compatible with the processes and tools that businesses use to analyze and predict consumer decisions.

An important issue when studying emotions is how to measure and interpret them. Much prior art related to the current invention addresses a purpose other than consumer research, mainly in the fields of medical diagnosis. For example: U.S. Pat. No. 6,947,790 by Gevins and patents referenced therein describe methods using electroencephalograph (EEG) measuring changes in a human subject's fundamental cognitive brain functions due to disease, injury, remedial treatment, for medical diagnosis purposes. U.S. Pat. No. 5,230,346 by Leuchter and related patents describe methods for determine brain conditions using EEG to obtain a diagnostic evaluation of brain diseases.

Although the importance of emotions in determining consumer behavior is now understood, there are few objective methods to collect and analyze such emotional responses. The few methods, that do exist, have been borrowed from medical or physiological fields and are not specifically adapted to meet the needs of today's marketing practitioners. The methods used throughout time to measure emotions in consumer research can be divided in two overall groups: Explicit measures such as verbal and visual self-report and implicit measures such as autonomic measures and brain imaging.

Self-report is the most commonly used explicit method for measuring emotions especially connected to consumer behavior. It is commonly used in focus group interviews, telephone surveys, paper-and-pencil questionnaires, online surveys, and instrument-mediated measurement systems using sliders or dials to capture moment-to-moment changes in emotional reactions. Responses measured include stated preferences among alternative products or messages, propensities to buy, likelihood of use, aesthetic judgments of product and packaging designs, moment-to-moment affective responses, and other predictions of likely future behaviors.

Although commonly used due to the low costs of acquiring the response data, self-report is difficult to apply to measuring emotions since emotions are often unconscious or simply hard to define causing bias to the reported emotions. They involve a long list of emotion adjectives and the rating can cause fatigue in the respondents which can damage the reliability. Furthermore self-report involves cognitive processing, which may distort the original emotional reaction.

Recently, researchers have begun measuring naturally occurring biological processes to overcome some of these problems of self-reporting. These measures are often referred to as implicit measures and can further divided into autonomic measures and brain imaging. These measurements has been used in consumer research as early as the 1920s mostly applied to measuring response to advertising.

Autonomic measures rely on bodily reactions that are partially beyond an individual's control. It therefore overcomes the cognitive bias linked to self-report. However most autonomic measures are conducted in a laboratory setting, which it is often criticized for, since it is considered out of social context. The most common autonomic methods include the measurement of facial expressions via Facial electromyography (EMG) or Facial Action Coding System (FACS) and Electrodermal reaction (EDR) or Skin conductance that measures activation of the autonomic nervous system.

U.S. Pat. No. 7,113,916 by Hill discloses a method to score visible facial muscle movements in video taped interviews and U.S. Pat. No. 20030032890 by Genco et al describes a method using facial electromyography (EMG) to measure facial muscle activity via electrodes placed on various locations on the face. The electrical activity is used to gauge emotion response to advertising. The main limitations of these methods are 1) they must be conducted in a laboratory setting with specific equipment 2) they requirements specialized skills not commonly available to market researchers to interpret the data 3) respondents are highly affected by the fact that they know they are being measured (physical contact of sensors) and therefore try to control muscle reactions (Bolls, Lang and Potter, 2001) 4) only a single metric is used to assess the impact of emotion on the presented stimulus, thus limiting the usefulness of the metric, and in the case of EMG it is nearly always impossible to reliability aggregate the results when measures are combined or averaged across a sample of consumers as different individuals have different baseline levels of activity that can bias such aggregation.

Electrodermal reaction (EDR) or Skin conductance measures activation of the autonomic nervous system which indicates ‘arousal’. The EDR measure indicates the electrical conductance of the skin related to the level of sweat in the eccrine sweat glands which is involved in emotion-evoked sweating and is conducted using electrodes. However this method requires a lot of experience and sensitive equipment. Furthermore EDR only measures the occurrence of arousal not the valence of the arousal, which can be both positive and negative. Another problem with using EDR are the individual variation and situational factors such as fatigue, medication etc, which makes it hard to know what you are measuring. U.S. Pat. No. 6,453,194 by Hill utilizes synchronized EDR signals to measure reactions to consumer activities and U.S. Pat. No. 6,584,346 by Flugger describes a multi-modal system and process for measuring physiological responses using EDR, EMG, and brainwave measures, but only for the purpose of assessing product-related sounds, such as the sounds of automobile mufflers.

Brain imaging is a new method in consumer research. The method has entered from neuroscience and offers the opportunity for interesting new insights. Emotions are pointed out as an area of specific relevance. However the method is extremely expensive, it requires expert knowledge and has severe technological limitations for experimental designs. Furthermore knowledge within neuroscience is still relatively young and therefore the complexity of the problems investigated must be relatively simple. The use in consumer research is so far relatively limited and thus are the examples of use related to measurement of emotions in consumer research. The most commonly applied methods from neuroscience are the Electroencephalography (EEG), Magnetoencephalography (MEG), Positron emission topography (PET), Functional Magnetic Resonance Imaging (fMRI) and U.S. Pat. No. 6,099,319 by Zaltman and U.S. Pat. No. 6,292,688 by Patton focus on the use of neuroimaging (positron emission tomography, functional magnetic resonance imaging, magnetoencephalography and single photon emission computer tomography) to collect brain functioning data while exposed to marketing stimuli and performing experimental tasks (e.g., metaphor elicitation).

Accordingly, there is a need for systems and methods of measuring consumer responses to external stimuli that avoid, or at least alleviate, these limitations and provide accurate and replicable measures of verbal, as well as non-verbal, responses. There is also a need to aggregate these measures across many samples to provide improved and more accurate analyses and research results than can be produced with prior art.

While prior art shows precise tools measuring physiological activities using methods such as facial electromyography (EMG), galvanic skin response (EDR) or neurological activity like fMRI-scanning used in consumer research, they are however have has significant limitations. They are impractical and very expensive if adapted to studies that demand large samples. The cost is high and the time carrying out these types of experiments is quite long. They also demand respondents to meet in specially adapted facilities or laboratories. They also limit the ability to generalize conclusions from a statistical viewpoint as they are in most case only applied to small samples.

Furthermore much prior art addresses methods for only acquiring emotional consumer research data, none specifically describe a complete system and method for measuring, computing, analyzing, and interpreting emotional responses to external stimuli such provided by aspects of the current invention.

### BRIEF SUMMARY OF THE INVENTION

It is one aim of the present invention to offer a method and a system related to the measurement and assessment of consumer's non-verbal response to a marketing stimulus, which is more practical and less expensive if adapted to studies that demand large samples.

It is another aim of the present invention to provide a method and a system related to the measurement and assessment of consumer's non-verbal response to a marketing stimulus, which is less time consuming than the known methods.

It is another aim of the present invention to provide a method and a system related to the measurement and assessment of consumer's non-verbal response to a marketing stimulus, which is can work across cultures without the need for adaptation to questionnaire design or scales.

It is another aim of the present invention to provide a method and a system related to the measurement and assessment of consumer's non-verbal response to a marketing stimulus, which can be easily carried out over a communication network such as the internet without the need of a special equipment except a standard home computer.

It is another aim of the present invention to provide a method and a system related to the measurement and assessment of consumer's non-verbal response to a marketing stimulus, which allows generalizing conclusions from a statistical viewpoint as they are applied to large samples.

It is another aim of the present invention to provide a method and a system related to the measurement and assessment of consumer's non-verbal response to a marketing stimulus, which allows a measure of emotional intensity to be calculated on a continuous scale without the need to ask any questions, attach any measurement device to a subject, or the use of a scoring system that needs to be applied by a human observer.

According to the invention, these aims are achieved by means of a method for assessing the impact of non-verbal responses of a respondent to a stimulus comprising the steps of:
presenting a reference stimulus to the respondent;
recording immediate non-verbal responses via an imaging device to said presented reference stimulus;
presenting a stimulus under test to the respondent;
recording immediate non-verbal responses via an imaging device to said presented stimulus under test;
presenting a questionnaire with questions on the stimulus to the respondent;
obtaining verbal responses to the questions;
transmitting the recorded image of said non-verbal responses to the reference stimulus and the stimulus under test across a communications network to an image processing unit and
after having received said images at said image processing unit automatically calculating emotion probabilities of the non-verbal responses of the respondent from said images; and
calculating a emotional intensity score derived from the emotional probabilities.

The method described in this invention bridges the gap between verbal self-report and autonomic non-verbal emotional response measurement methods while adding an objective and scientific analyze of non-verbal response to a stimulus. It is a scientific method, enabling marketers to effectively track consumers' conscious and unconscious feelings and reactions about brands, advertising, and marketing material. It has numerous advantages for businesses in that it is fast and inexpensive, and given its simplicity, is applicable to large samples, which are a necessary condition for valid and statistical inference. This approach reduces significantly the cost of making more accurate decisions and is accessible to a much larger audience of practitioners than previous methods. It is objective and commercially practical.

Advantageously the stimulus presented to the participant is from the group comprising of an advertisement represented by one or a combination of images, video, text, or sound, new or existing products, new or existing web-pages, marketing and sales material, presentations, speeches, newspaper articles, movies, music, videos games, logos, store fronts, graphical identities of new or merged companies, or financial charts.

As an additional advantage the survey can be uploaded to a data processing unit and the respondent answers said survey as a stimulus over said communications network from a local computer of the respondent, which then allows reaching a significantly larger number of participants. This can be done in that a respondent receives a link via an email and by clicking on the link in said email, he is directed to an online survey with the inventive method.

Recording a facial image of said respondent by a webcam as an imaging device is performed and the recorded image is transmitted over the internet as communication network gives an additional advantage.

To reduce bandwidth or storage requirements the captured image of said non-verbal response can be further processed before storing or sending it to said image processing unit. This step can comprise an image compression or identifying a region of interest related to the non-verbal response within the image.

Said non-verbal response is one or a combination of an emotional response or visual attention response, wherein said non-verbal response is an emotional response, can be performed by a number of state-of-the-art algorithms that :
compute a set of features derived from the image
use machine learning algorithms to classify image to produce a set a of probabilities of specific emotional states or a single probability of a measure directly linked to emotion such as valence.

The predicted classification probabilities can represent basic emotions such as happiness, surprise, fear, and disgust sadness or any other emotional state or measure such as valence in both positive and negative terms.

In order to allow for interpretation and analysis a single emotion intensity score is calculated from the emotion probabilities. Two methods are preferred. Firstly a weighted sum of emotion probabilities called Emotion Intensity Score Absolute or a weighted sum of the difference between the reference stimulus and stimulus under test called Emotion Intensity Score Relative.

Emotion Intensity Score Absolute=w1×Emotion Probability of Stimulus 1+w2×Emotion Probability of Stimulus 2+ . . . +wn×Emotion Probability of Stimulus n

Emotion Intensity Score Relative=w1×(Emotion Probability of Stimulus 1−Emotion Probability of Reference 1)+w2×(Emotion Probability of Stimulus 2−Emotion Probability of Reference 2)+ . . . wn×(Emotion Probability of Stimulus n−Emotion Probability of Reference n)

The weighting factors w1, w2, . . . wn can be determined in a number of ways, including by experimentation and the theory of marketing communication models, however the preferred method involves optimizing the weighting coefficients by maximizing the correlation between the Emotion Intensity Score and response data linked to long term memory effects which can come from brain imaging or any form of brain activity data.

An analysis of verbal responses by said determined non-verbal emotion intensity score can be reported for analyzes reasons. It can further be identified how determined the emotion intensity score associates with dependent variables of the presented stimulus, wherein the dependent variables are liking, adoption, or purchase intention.

A facial image of the respondent can continuously be recorded from the moment the respondent is presented with the reference stimulus or directly the stimulus under test to the instance when the respondent ends the survey or for specific configurable periods and continuously transmitted to the image processing unit over the communication network for calculating the predicted classification probabilities and emotion intensity score. In addition, if the stimulus is a video or dynamic media content, embedded cue points in such media can be used to associate the exact recorded image of the non-verbal responses of the respondent to the correct frame or moment when the stimulus was presented. Alternatively, if embedded cue points are not available, timestamps can be used instead.

Before the method is started a question for calibration can be asked in order to improve the model estimation of predicted probabilities of facial descriptions, wherein respondents are probed with images of people electing facial expressions and are asked to categorize those based on a predetermined list of facial expressions and for the non-verbal response an automated facial expression classification system generates a set of predicted emotion probabilities for each respondent.

The classification probabilities, computed emotion intensity score, and verbal responses can be merged based on timestamps or cue points and an id which can be unique to the respondent in a new data file of the merged data, which is stored in a data processing unit for further analysis methods employing descriptive, econometrics methods, multivariate techniques, data mining techniques, wherein descriptive statistics such as contingency tables are generated for each question utilized in the questionnaire.

According to the invention, these aims are achieved as well by means of an independent method for assessing the impact of non-verbal responses of a respondent to a stimulus comprising the steps of:
presenting a reference stimulus to the respondent on a computer;
presenting a stimulus under test to the respondent on a computer;
recording an immediate non-verbal responses to said presented stimuli via an imaging device connected to said computer;
presenting a questionnaire with questions on the stimulus under test to the respondent on said computer;
obtaining verbal responses to the questions; and sending the verbal responses across a communications network to a data processing unit;
transmitting the recorded image of said non-verbal responses across said communications network to an image processing unit and
after having received said images at said image processing unit automatically calculating a distribution of probabilities of one or a combination of an emotional state, a visual attention, a demographics of the face or a posture of the non-verbal response from said images;
determining a emotion intensity score from combining said predicted classification probabilities; and sending said predicted classification probabilities and emotion intensity score from said image processing unit to said data processing unit; and
reporting an analysis of verbal responses by said determined emotion intensity score at the data processing unit.

According to the invention, these aims are achieved as well by means of an independent method for assessing the impact of non-verbal responses of a respondent to a stimulus comprising the steps of:
presenting a reference stimulus to the respondent;
presenting a stimulus under test to the respondent;
recording immediate non-verbal responses via an imaging device to said presented stimuli;
presenting a questionnaire with questions on the stimulus under test to the respondent;
obtaining verbal responses to the questions;
transmitting the recorded image of said non-verbal responses across a communications network to an image processing unit and
after having received said images at said image processing unit calculating emotional probabilities of the non-verbal responses of the respondent by using statistical inferences of the received images; and
determining a emotion intensity score from said predicted classification probabilities.

The inventive can use an automated expression classification system for the generation of a set of predicted emotion probabilities for each respondent as statistical inferences. Preferably facial expressions are used.

According to the invention, these aims are achieved as well by means of an independent system for assessing the impact of non-verbal responses of a respondent to a stimulus, said system comprising
a data processing unit with a reference stimulus, a stimulus under test and questions for said stimulus to be presented to the respondent;
an image device for recording an immediate non-verbal response of said respondent to said presented stimulus under test;
means for transmitting the recorded image of said non-verbal response across a communications network to an image processing unit;
said image processing unit comprising means for automatically calculating emotion probabilities of the non-verbal response(s) of the respondent from said images employing statistical techniques; and
said data processing unit comprises means for determining emotion intensity score from said emotion probabilities and means for reporting at said a data processing unit an analysis of verbal response(s) by determined emotion intensity score.

### BRIEF DESCRIPTION OF THE DRAWINGS

The invention will be better understood with the aid of the description of an embodiment given by way of example and illustrated by the figures, in which:

FIG. 1 represents one embodiment of a system in which the method steps can be carried out across a communications network as an online survey.

FIG. 2 is an overall flow chart of one embodiment showing the major steps to conduct an online survey in applying the method to assess emotional impact to a marketing stimulus.

FIG. 3 is a detailed flow chart showing the step-by-step actions used to generate a survey as illustrated in FIG. 2.

FIG. 4 is a detailed flow chart showing the step-by-step actions used to conduct a survey as illustrated in FIG. 2.

FIG. 5 is a detailed flow chart showing the step-by-step actions used to determine non-verbal response probabilities as illustrated in FIG. 2.

FIG. 6 is a detailed flow chart showing the step-by-step actions used to extract survey data as illustrated in FIG. 2.

FIG. 7 is a detailed flow chart showing the step-by-step actions used to analyze the survey data as illustrated in FIG. 2.

FIG. 8 is a system flow diagram of one embodiment describing how the method can be executed through an online web survey and across a communications network.

FIG. 9 illustrates how non-verbal emotional intensity score in absolute terms can be graphically reported over a entire sample of respondents over time periods.

FIG. 10 illustrates how non-verbal emotional probabilities can be graphically reported for the average across a single time period of a group of respondents.

FIG. 11 illustrates how non-verbal emotional intensity score in relative terms referenced to the reference stimulus can be graphically reported over a entire sample of respondents over time periods.

FIG. 12 illustrates how non-verbal emotion probabilities can be graphically reported over time periods of the stimulus of the average of a group of respondents.

FIG. 13 illustrates how dominant non-verbal emotion probabilities can be graphically reported over time periods of the stimulus.

FIG. 14 illustrates how non-verbal emotion probabilities can be graphically reported over time periods of the stimulus of a single respondent.

### DETAILED DESCRIPTION OF POSSIBLE EMBODIMENTS OF THE INVENTION

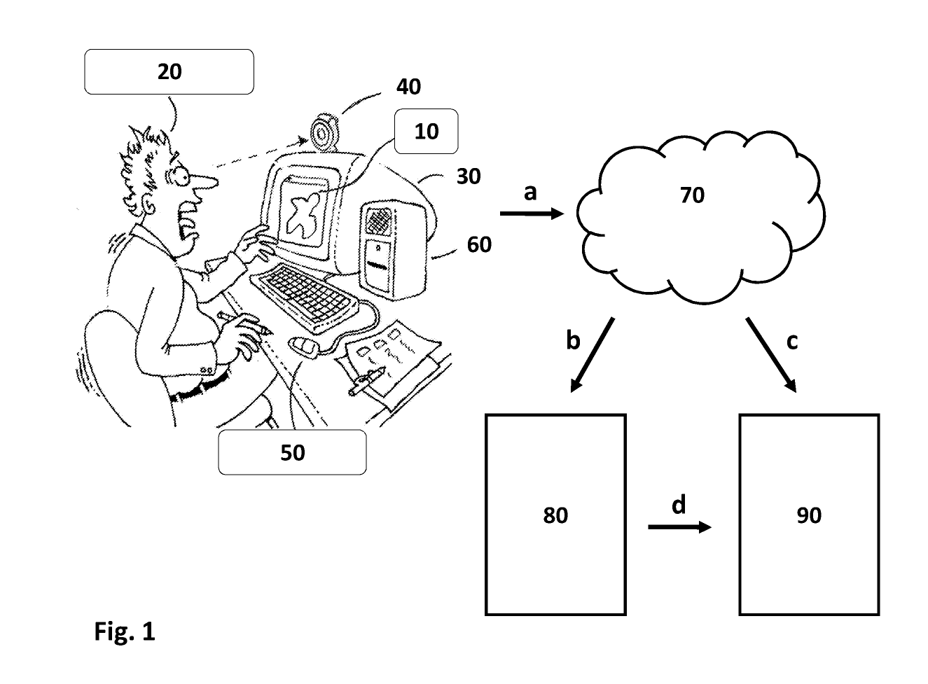

FIG. 1 represents one embodiment of a system in which the method steps can be carried out as an online of offline survey. A stimulus 10 is presented to respondent 20, generally recruited to participate in the survey as belonging to a particular target market population. The stimulus is displayed on a display unit 30 while an image capture device 40, such as a webcam, captures non-verbal responses of the respondent such as facial expressions or head and eye movements while he is exposed to the stimulus 10 and answers questions of the survey. The survey respondent needs no special instructions while performing the survey in relation to his non-verbal response being imaged i.e. does not need to look into the camera, he is free to move his body or head, and he can touch his face, etc.

After being exposed to the stimulus 10, the verbal responses to questions of the survey can be recorded using an input device 50 such as a keyboard or mouse. The recorded non-verbal and verbal responses can be stored directly on a local storage device 60 such as a memory of the computer or directly and immediately transmitted or sent across a communications network 70 such as the Internet to servers for further processing (step a). The image of the non-verbal response is sent to an image processing server unit 80 (step b), while the verbal response data is sent to a data processing server unit 90 (step c). Directly after having received said images at said image processing unit predicted classification probabilities of the non-verbal responses of the respondent from said images are automatically calculated. When the images are received from the image capturing device, the automatic calculations are done continuously with the received images. Both the image and data processing server units 80, 90 can be integrated in the same server unit having software means for analyzing the non-verbal and the verbal responses and calculating the results. Finally the predicted classification probabilities of the non-verbal response are sent from the image processing unit to the data processing unit for further analysis (step d).

The preferred embodiment of this invention is an online survey intended to test any marketing element, such as concept, print advertising, in-store display, or video advertising. However, alterative embodiments and applications are envisaged, such as one-to-one interviews, off-line surveys, kiosks, mobile surveys, focus groups, and webex surveys for different types of stimulus which may not be suitable for online surveys.

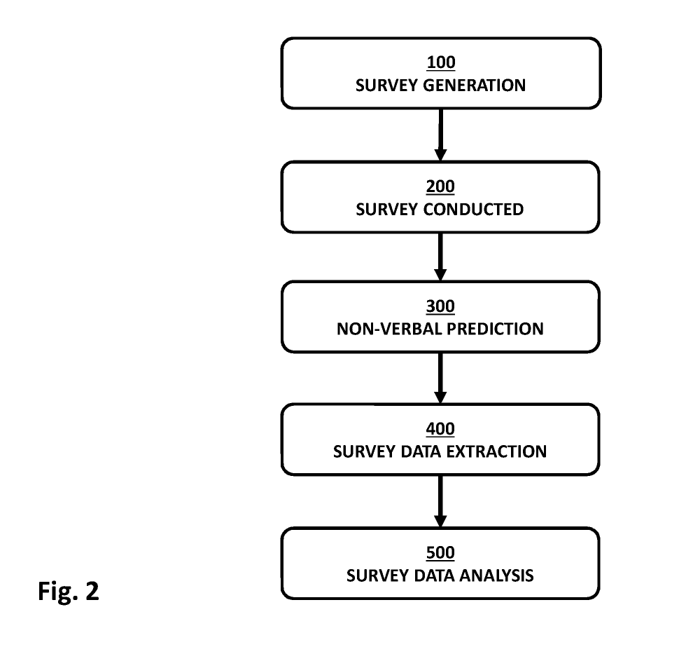

In the case of the preferred embodiment of an online survey, and referencing FIG. 2, it shows the overall steps of the inventive method. A survey can be generated in step 100 that is conducted in step 200. The non-verbal responses of the survey are predicted in step 300 via images taken during the survey of the respondent. The predicted non-verbal responses can be merged with the verbal responses of the survey in step 400 to permit data analysis in step 500 of the survey response data.

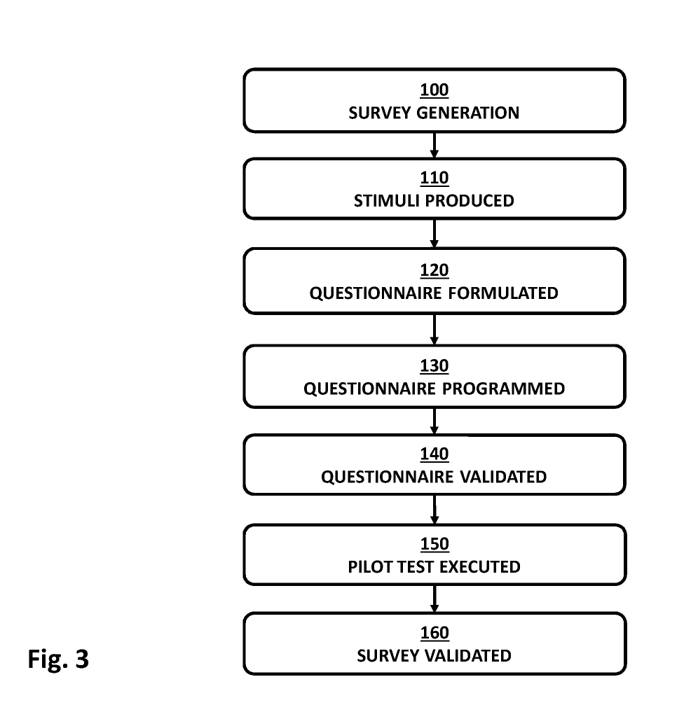

FIG. 3 illustrates the steps of how an online survey can be generated 100. First stimuli are produced in 110. Stimuli can take form of video, audio, pictures (images), text and any combination of those. Stimuli are produced to test a hypothesis. Examples are any advertising, marketing, or sales material (but not restricted to those) consisting of video, audio, pictures (images), text of advertisement, concepts, new or existing products, new or existing web-pages, trends, graphical identity of new or merged companies. Next in 120 a questionnaire is formulated based on the hypothesis to test. The questionnaire can include open and closed ended questions. Questions can utilize likert type scales, best worst scales and can be multiple or single choice. In 130 the questionnaire is programmed for online and off-line testing. Stimuli material can be programmed to be presented in randomized or listed order to respondents. In 140 the questionnaire can be validated if required. This can be via an initial test with one to one interviews carried out in order to assess validity of questionnaire, scales and stimuli presented. In 150 a pilot test of the survey is conducted. If the questionnaire is validated, a pilot test with a small sample of respondents can be carried out. The pilot mimics the actual survey in terms of questionnaire, method (web or face to face), stimuli presented and target audience. In 160 the final survey can be validated. Based on the results of 150, eventual ratifications can be provided to the overall survey.

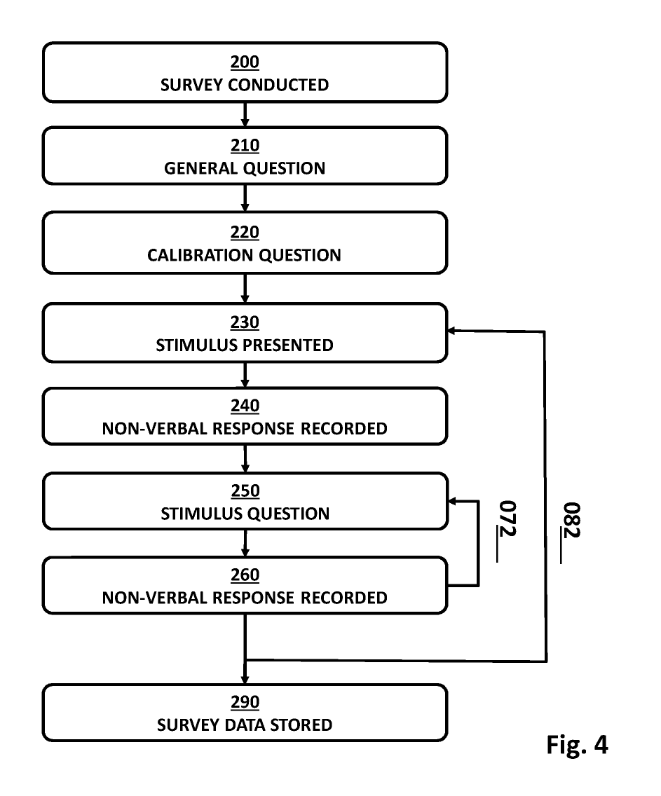

FIG. 4 illustrates how the survey that can be generated is carried out in its full scale (both in term of its content and in term of its target audience). In 200 the survey is started. This can be by participants receiving a link via an email. By clicking on the link they are directed to the online survey. Prior to answering the survey, participants are asked via a popup screen or window message or by any other UI element, if they agree or disagree with the procedure of recording images during the survey of their non-verbal responses. If they agree respondents can be provided with an introductory text that allows them in a short and easy manner, to set up their computer web camera, although this step can be optional. The survey then functions as any other online survey, where no additional software needs to be installed, with the only difference being that images of the respondent are recorded during the survey. Images can be recorded continuously from the moment the respondent is presented with a stimulus to the instance when the respondent ends the survey or for specific configurable periods.

In 210 general questions can be asked (non intrusive) with the aim to make familiar the respondent with the questionnaire. In 220 a reference stimulus is shown to the respondent. This is an optional step in order to allow better descriptive statistics to be developed using the emotion probabilities, respondents are shown a blank image or a images for a fixed length of time before the stimulus under test is shown. In 230 the stimulus under test is presented to the respondent while in 240 an image of his immediate reaction to the reference stimulus and the stimulus under test are recorded and can be stored locally (such as in FIG. 1 on a local storage device 60) or on a server 80, which can be secured using stand encryption standards or techniques. The captured image can be further processed before transmitting the captured image(s) and/or before storing to reduce bandwidth or storage requirements. This processing can include image compression, such as JPEG, PNG or identifying a region of interest related to the non-verbal response within the image. In 250 the respondent can be asked a question on the stimulus presented. Again in 260 the image of the non-verbal response is recorded while the respondent is answering the question. In 270, the steps 250 and 260 can be repeated for as many times is necessary for hypothesis testing. In step 280, steps 220 to 270 can be repeated per stimuli. Finally data collected during the survey are stored in 290 either locally (such as a local storage device 60) or by sending the data across a communications network such as the internet to a server 80. In the case data is stored on a server the images of non-verbal responses can be stored on a separate server 80 to the data concerning the verbal responses to the survey test such as a server 90.

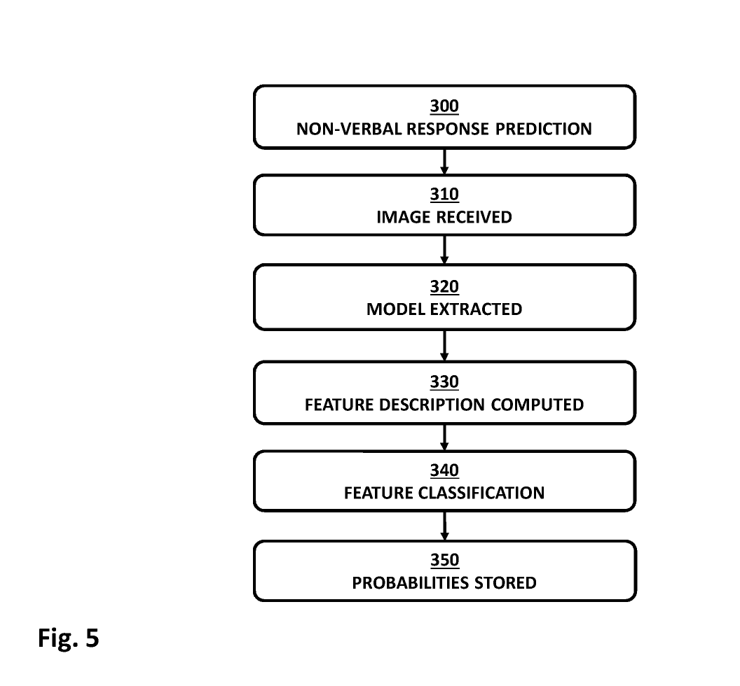

FIG. 5 illustrates how an automated non-verbal response classification system generates a set of predicted probabilities for each respondent, based on the viewing of the proposed stimuli. The aim of 300 is to classify the non-verbal response(s) by class. The non-verbal response can be any response that are communicated through facial expressions, head or eye movements, body language, repetitive behaviors, or pose which can be observed through an image or series of images of the respondent. The most common non-verbal responses used in an online survey are emotions and visual attention expressed by spontaneous facial expressions or eye and head movements. In addition this step can also comprise of classifying demographic characteristics of the respondent such as gender, age, or race. The process starts in 310 by the system receiving an image. In the embodiment of an online survey, the image is received across a communications system, such as the internet 70, however it is not limited to this means of transmission. In an offline survey, for example, the images may be transferred by means of a portable storage device for later use. The image may also be acquired or obtained from locally stored images on a file system, or by image capture devices connected directly to the system.

In 320 the image can be processed to build a model based representation. The aim of this process is to map features of the respondent such as the face or body present in the image to a model based representation, which allows further descriptive processing in 330. Faces and bodies are highly variable, deformable objects, and manifest very different appearances in images depending on pose, lighting, expression, and the identity of the person and the interpretation of such images requires the ability to understand this variability in order to extract useful information. There are numerous methods to convert a deformable object, such as the face, into a model based representation, however in 310 we prefer the use Active Appearance Models (AAMs), although other model based representations are possible.

In 330 the model based represented in 320 can be processed to extract a feature description. The aim of this processing is to generate a measurable description, based on movements, presence of features, and visual appearance found in the model, which can be relevant visual cues to the classification step of 340. Numerous techniques can be employed to extract the feature description, however in the case of building feature description for emotion classification we prefer the use of a combination of Facial Actions Unit Coding System (FACS), Expression Description Units (EDU), and AAM Appearance Vectors. However step 320 is not limited in any way these preferred feature descriptions. The processing of 320 can be a single processing stage or divided into multiple stages. In the case the non-verbal response is an emotional response, three stages are preferred, where the first stage involves computing the measures coming from the FACS, the second stage computes a set of configurable measures such as EDU, and a third set of measures important from the human perceptual point of view are a set of measures representing the appearance of the face. The feature description is then passed to 340 for classification.

The aim of 340 is to classify the feature description computed in 330. Many consumer research applications are interested to know emotions such as happiness, sandiness, anger, etc. However 330 is not in any way limited to emotion classification, it can also include classification of visual attention or any demographics of the face such as gender, age or race. The classification can be performed with numerous methods such as support vector machines, neural networks, decision trees, or random forests. In this case, discrete choice models are preferred for expression classification, as they have been shown to give superior accuracy performance.

In the presented method, it is automatically calculated a distribution rather than unique categorization of the perceived emotional responses of each respondent. Thereby a probability of emotion per image is used employing statistical techniques to associate the emotion probability to impact on the response to a presented stimulus. In contradiction to some prior art documents the inventive method does not rely on empirical methods (such as lookup tables or similar), but uses only statistical inferences on estimated emotional probabilities of the received images instead of scores based on the presence of emotional cues. The present approach is therefore not only different in this respect, but superior as it is more objective, precise, and benefits from large sample sizes by using statistical inference on estimated emotional probabilities, instead of scores based on the presence of emotional cues.

The output of 340 are the predicted probabilities of the respondent image. These probabilities are then used to compute the Emotion Intensity Score (EIS) using a weighted sum of the predicted probabilities:

EIS=w1×Probability of Happiness+w2×Probability of Surprise+ . . . wn×Probability of Selected Emotion

The EIS can be further segmented into type such as groups such as Positive and Negative by leaving out certain predicted probabilities for example:
EIS (Positive)=w1×Probability of HappinessAdditional logic can also be used to improve the reliability of the EIS calculation by condition logic applied to the change in of the probabilities of emotions. For example if increasing surprise is followed by increasing happiness, then the surprise may be counted towards EIS (Positive). The weights used to calculate EIS can be found in numerous ways, however it is preferred to find the weights by solving an equation that maximizes the statistical correlation between the calculated EIS and another set of measures related to brain activity such as long term memory retention.

The output of 340 and 350 is the the intended variables to be classified and used in analysis. These variables can be then stored in 360, in any means appropriate, such as in a spreadsheet on the local file system or in a database. Once stored, they then can be downloaded and merged with the verbal data from the survey to be further processed.

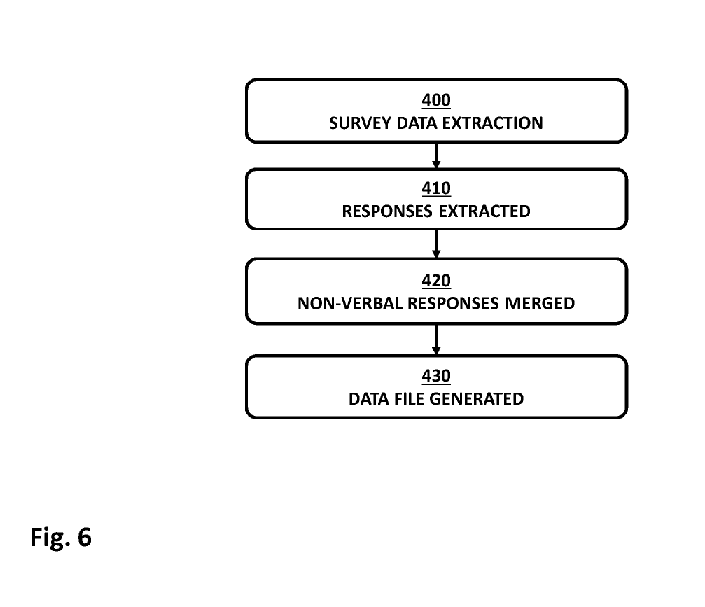

FIG. 6 illustrates in 400 how survey data can be extracted and prepared for analysis. In 410 data containing the verbal responses and the classification probabilities of their non-verbal responses can be extracted from the server(s). The classification probabilities can represent emotion, visual attention, age, race, gender, etc as described in step 340. In 420 the classification probabilities and verbal responses can be merged based on timestamps or cue points and respondent IDs. A new data file of the merged data can be stored in 430 which is ready for analysis by employing descriptive, econometrics methods, multivariate techniques, or data mining techniques.

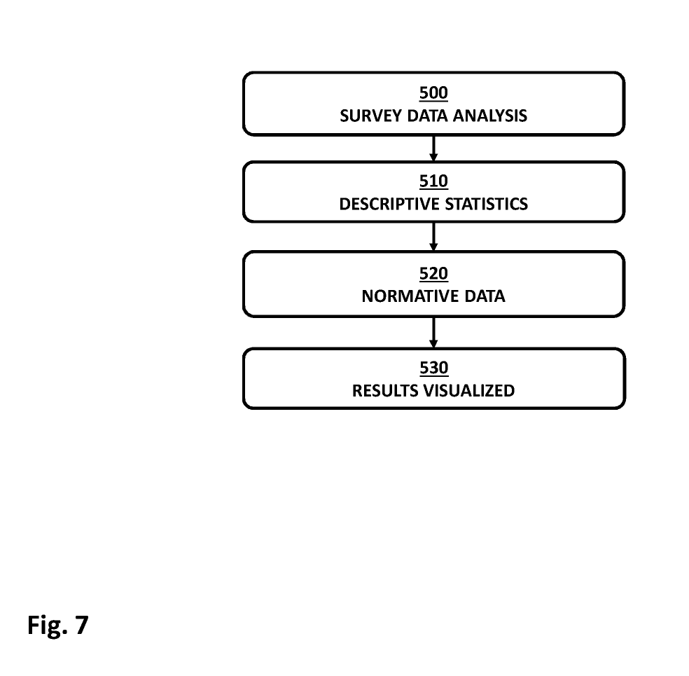

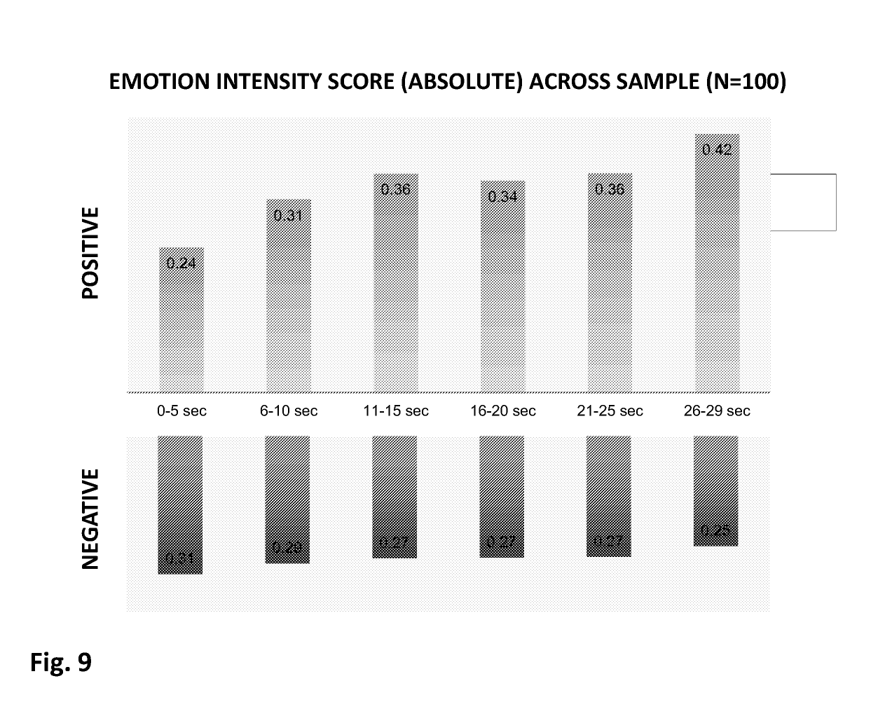

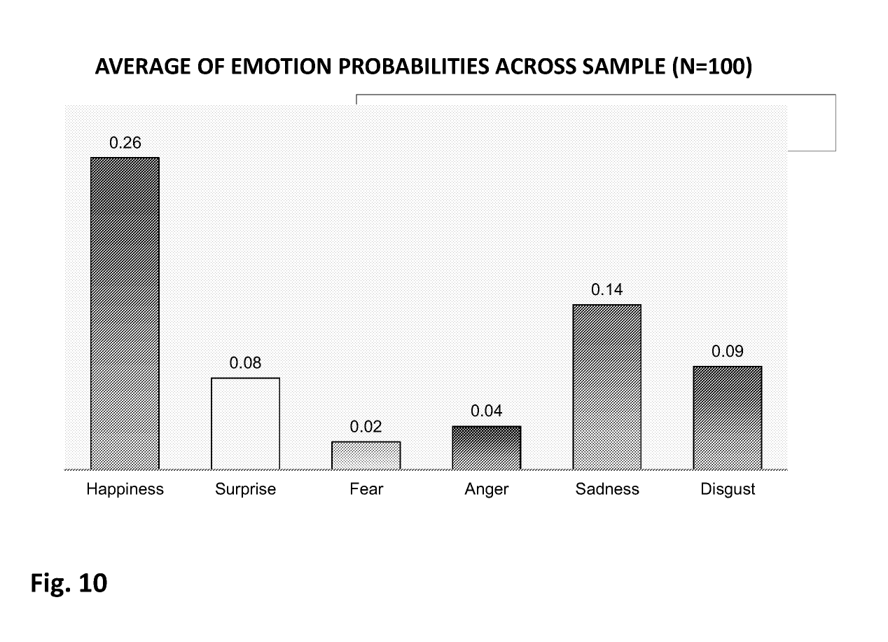

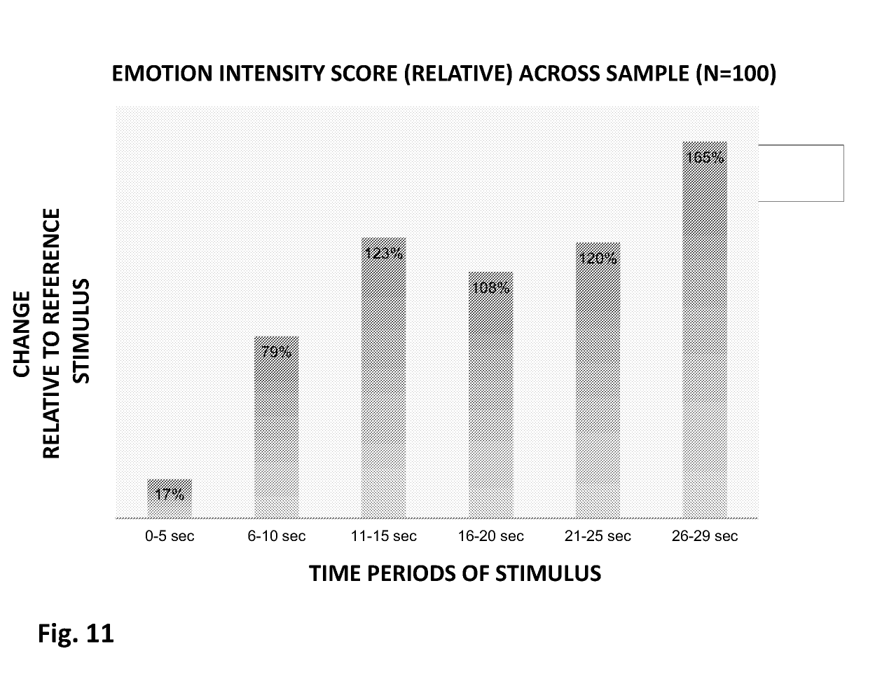

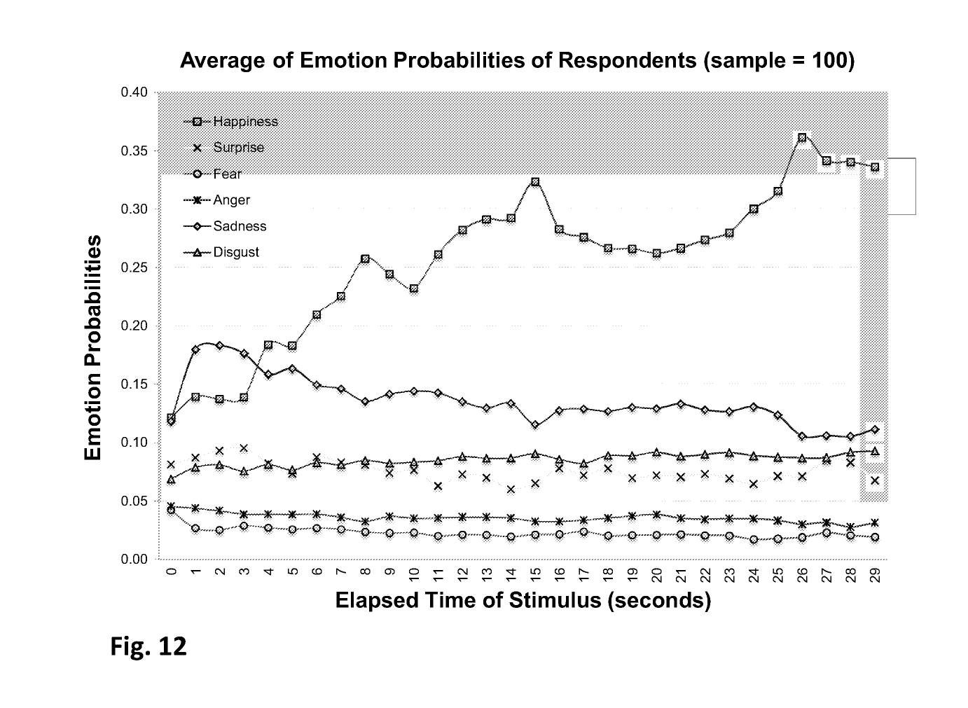

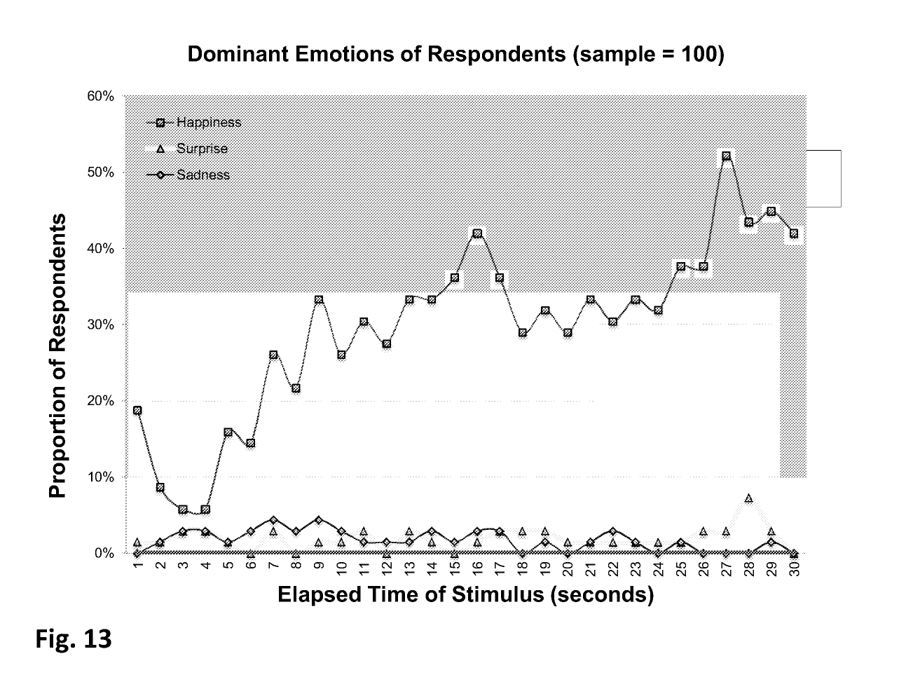

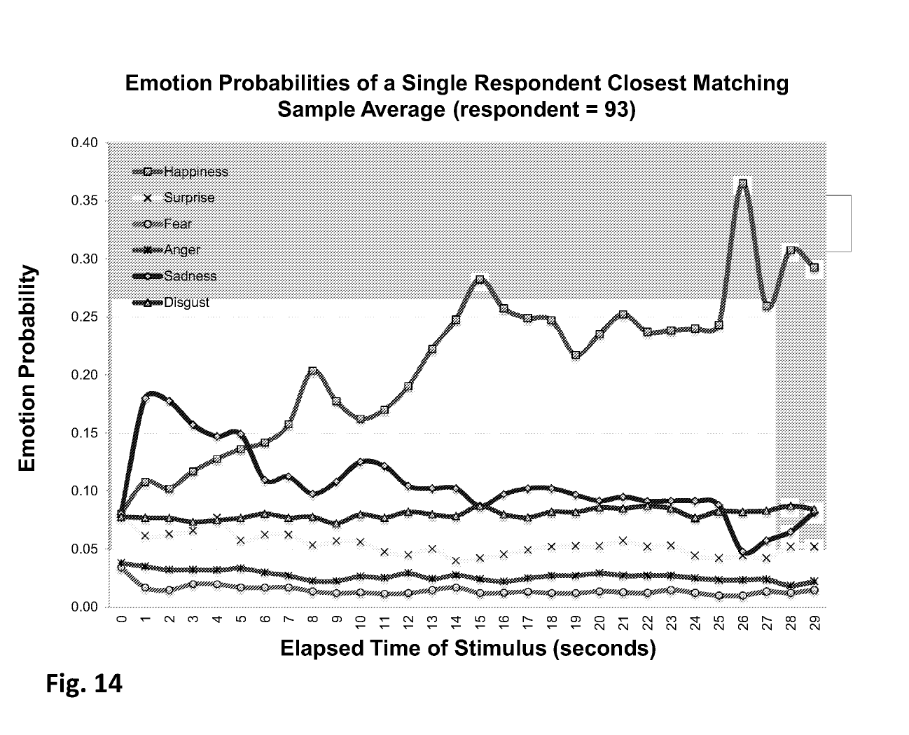

In 500 data analysis is performed on the merged data as illustrated in FIG. 7. In 510 descriptive statistics such as contingency tables can be generated for each question utilized in the questionnaire. Charts and tables of predicted probabilities and emotion intensity scores of non-verbal responses are also generated. In 520 the outputs of 510 are compared versus normative data on the descriptive statistics and visualized in 530. FIGS. 9, 10, 11, 12, 13, and 14 illustrate how non-verbal emotional probabilities and the emotion intensity score can be visualized over a) time periods of the stimulus b) for a single respondentc) average of all respondents over all images. Other visualizations in different formats can be envisaged depending on the type of stimulus used in the survey.

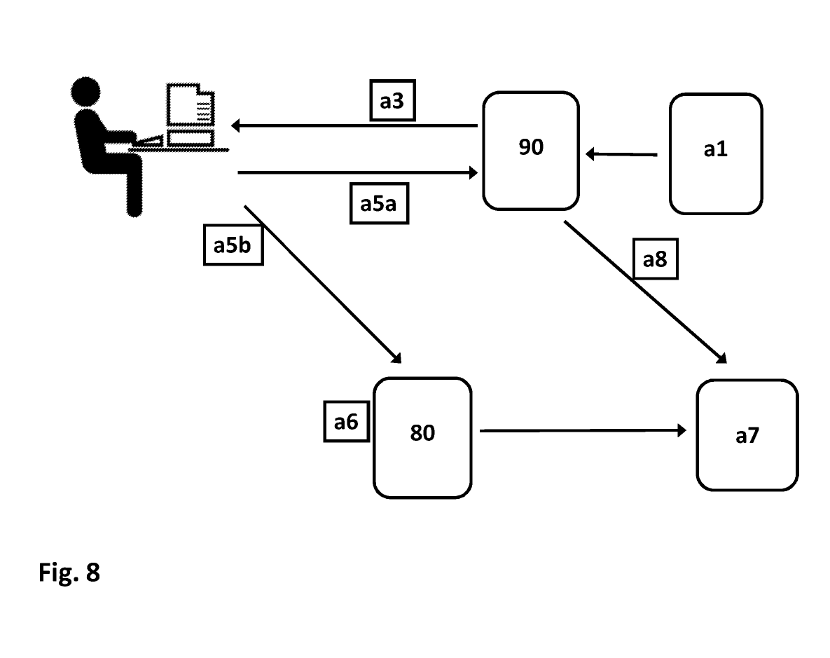

FIG. 8 is a system flow diagram of one embodiment describing how the method can be executed through an online or offline web survey and across a communications network:

a1. Design; Programming of based questionnaire: A questionnaire can be programmed for the online or offline survey. Depending on the type of survey, a variety of different programming languages can be used such as html, flash, php, asp, jsp, javascript, or java although the choice of programming language is not limited in anyway to these examples.

a2. Deployment of survey: In the case of an online survey, i.e. where the respondent answers the survey on the internet, the survey can be uploaded to a server. In the case of an offline survey the survey can be deployed directly on the computer of the respondent.

a3. Invitation; respondents can be invited to answer the online or offline survey in which the stimuli material is presented: Respondents can be contacted via a variety of methods such as email, telephone or letter to take part in the survey. For online panels this mostly happens via email. However other means can be used. As the survey can be carried out offline and can be a face to face interview, the step functions in both situations.

a4. Non-verbal response prediction reference: An optional step can be used where respondents are shown a reference stimulus before showing the stimulus under test. The respondents non-verbal response can be recorded as a sequence of images captured using an imaging device such as a web camera, and

a5. The respondent answers the questionnaire: The respondents non-verbal response can be recorded as a sequence of images captured using an imaging device such as a web camera. The respondent's verbal responses can be recorded using a mouse, key board, or microphone, or directly recorded by an interviewer in the case of a face-to-face interview. The verbal answers to the questionnaire (a5a) can be stored in server 90. Images of non-verbal responses can be stored server 80 (a5b). Server 80 and Server 90 can be the same or a different server or different software modules at the same server.

a6. An automatic non-verbal recognition system can be used to compute predicted probabilities of non-verbal responses. In the case that the non-verbal response is an emotional response, the predicted probabilities can represent basic emotions such as happiness, surprise, fear, and disgust sadness or any other emotional state. Other non-verbal responses can also include visual attention and posture, but is not limited in any way to these examples.

a7. Data file is automatically produced with vector of predicted probabilities and emotion intensity scores per respondent per stimuli presented. A data file is now ready for analysis. It can contain all variables from the questions used in the survey with the vector of predicted probabilities for the non-verbal responses for the questions or stimuli where the non-verbal responses have been captured.

Alternative Embodiments: Although this invention has been described with particular reference to its preferred embodiment in consumer research, it is envisaged by the inventors in many other forms such as :
One to one interviews
Mobile surveys
Offline surveys
Retail kiosks
Focus groups
Webex survey (with and without interviewer)

In addition, the method is applicable to any domains or applications, where analyzing the impact of human emotional response(s) to a stimulus is important in a decision making context. The following are intended as examples only, not an exhaustive list:
Copy testing
Print ads
TV ads
Direct mail
Newspaper
Radio
Outdoor
Usability testing
Product
Packaging
Web site
Customer experience
Customer satisfaction
Human resources
Employee satisfaction
Negotiation training
Hiring
Sales force training
Brand and Strategy
Design and positioning
Logo testing
Brand equity
Pricing
Finance
Risk management

The method described in this invention bridges the gap between verbal self-report and autonomic non-verbal emotional response measurement methods while adding an objective and scientific analyze of non-verbal response to a stimulus. It is a scientific method, enabling marketers to effectively track consumers' conscious and unconscious feelings and reactions about brands, advertising, and marketing material. It has numerous advantages for businesses in that it is fast and inexpensive, and given its simplicity, is applicable to large samples, which are a necessary condition for valid and statistical inference. This approach reduces significantly the cost of making more accurate decisions and is accessible to a much larger audience of practitioners than previous methods. It is objective and commercially practical.

Major advantages over current methods include:
Suitable for large scale survey sampling without need for expensive equipment.
Deployable outside of the laboratory environment.
Applicable cross-culturally and language independent.
Measurement of emotional responses are free from cognitive or researcher bias.
Gives objective measurements and analysis without the need for highly trained personnel or expert domain knowledge in emotion measurement.

1. A method for assessing the impact of non-verbal responses of a respondent to a stimulus comprising the steps of:
presenting a reference stimulus to the respondent;
recording immediate non-verbal responses via an imaging device to said presented reference stimulus;
presenting a stimulus under test to the respondent;
recording immediate non-verbal responses via an imaging device to said presented stimulus under test;
presenting a questionnaire with questions on the stimulus under test to the respondent;
obtaining verbal responses to the questions;
transmitting the recorded image of said non-verbal responses to the reference stimulus and the stimulus under test across a communications network to an image processing unit and
after having received said images at said image processing unit automatically calculating emotion probabilities of the non-verbal responses of the respondent from said images; and
calculating an emotional intensity score derived from the emotional probabilities.

2. The method according to claim 1, wherein the stimulus is from the group comprising of an advertisement represented by one or a combination of images, video, text, or sound, new or existing products, new or existing web-pages, marketing and sales material, presentations, speeches, newspaper articles, movies, music, videos games, graphical identities of new or merged companies, or financial charts.

3. The method according to claim 1, wherein a survey is uploaded to a data processing unit and the respondent answers said survey as a stimulus over said communications network from a local computer of the respondent.

4. The method according to claim 1, wherein recording a facial image of said respondent by a webcam as an imaging device is performed and the recorded image is transmitted over the internet as communication network.

5. The method according to claim 1, wherein the captured image of said non-verbal response is further processed before storing or sending it to said image processing unit to reduce bandwidth or storage requirements.

6. The method according to claim 5, wherein the steps comprise image compression or identifying a region of interest related to the non-verbal response within the image.

7. The method according to claim 1, wherein said non-verbal response is one or a combination of an emotional response or visual attention response.

8. The method according to claim 7, wherein said non-verbal response is an emotional response, three steps are performed
computing the measures coming from the Facial Action Coding System (FACS),
computing a set of configurable measures called Expression Descriptive Units (EDU), and
setting measures representing the appearance of the face.

9. The method according to claim 1, wherein the predicted classification probabilities represent basic emotions such as happiness, surprise, fear, and disgust sadness or any other emotional state.

10. The method according to claim 1, wherein an emotion probability per image is calculated employing statistical techniques.

11. The method according to claim 1, wherein the step of calculating emotion probabilities of the non-verbal responses comprises the steps of converting the image of the respondent into a model based representation, extracting a feature description of said model based representation and generating a measurable description, based on movements, presence of features, and visual appearance found in the model.

12. The method according to claim 1, wherein the respondent receives a link via an email and by clicking on the link in said email, he is directed to an online survey with the method steps of claim 1.

13. The method according to claim 1, comprising the step of reporting an analysis of verbal responses by said determined non-verbal segments.

14. The method according to claim 1, wherein a facial image of the respondent is recorded continuously from the moment the respondent is presented with a stimuli to the instance when the respondent ends the survey or for specific configurable periods.

15. The method according to claim 1, wherein before the method a question for calibration is asked in order to improve the model estimation of predicted probabilities of facial descriptions, wherein respondents are probed with images of people electing facial expressions and are asked to categorize those based on a predetermined list of facial expressions.

16. The method according to claim 1, wherein for the non-verbal response an automated facial expression classification system generates a set of predicted emotion probabilities for each respondent.

17. The method according to claim 16, wherein a combination of Facial Actions Unit Coding, Expression Description Units, and AAM Appearance Vectors are used in the automated facial expression classification system.

18. The method according to claim 16, wherein the emotion probabilities and verbal responses are merged based on timestamps or cue points and respondent ID in a new data file of the merged data, which is stored in a data processing unit for further analysis methods employing descriptive, econometrics methods, multivariate techniques, data mining techniques.

19. The method according to claim 18, wherein descriptive statistics such as contingency tables are generated for each question utilized in the questionnaire.

20. A method for assessing the impact of non-verbal responses of a respondent to a stimulus comprising the steps of:
presenting a reference stimulus to the respondent on a computer;
presenting a stimulus under test to the respondent on a computer;
recording an immediate non-verbal responses to said presented reference stimulus and stimulus under test via an imaging device connected to said computer;
presenting a questionnaire with questions on the stimulus under test to the respondent on said computer;
obtaining verbal responses to the questions; and sending the verbal responses across a communications network to a data processing unit;
transmitting the recorded image of said non-verbal responses across said communications network to an image processing unit and
after having received said images at said image processing unit automatically calculating a distribution of probabilities of one or a combination of an emotional state, a visual attention, a demographics of the face or a posture of the non-verbal response from said images;
determining a emotion intensity score from combining of said predicted classification probabilities; and sending said predicted classification probabilities and emotion intensity score from said image processing unit to said data processing unit; and
reporting an analysis of verbal responses by said determined emotion intensity score at the data processing unit.

21. The method according to claims 20, wherein the non-verbal responses are communicated through facial expressions, head or eye movements, body language, repetitive behaviors, or pose which is observed through said image or series of said images of the respondent.

22. The method according to claims 20, wherein the step of calculating an emotional intensity score comprises a weighted sum of emotion probabilities or a weighted sum of the difference between the reference stimulus and stimulus under test.

23. The method according to claims 20, comprising the step of utilizing said calculated probabilities as clustering variables.

24. The method according to claim 20, wherein a probability distribution per received image is calculated employing statistical techniques.

25. A method for assessing the impact of non-verbal responses of a respondent to a stimulus comprising the steps of:
presenting a reference stimulus to the respondent;
presenting a stimulus under test to the respondent;
recording immediate non-verbal responses via an imaging device to said presented reference stimulus and the stimulus under test;
presenting a questionnaire with questions on the stimulus under test to the respondent;
obtaining verbal responses to the questions;
transmitting the recorded image of said non-verbal responses across a communications network to an image processing unit and
after having received said images at said image processing unit calculating emotional probabilities of the non-verbal responses of the respondent by using statistical inferences of the received images; and
determining an emotion intensity score from said probabilities.

26. The method according to claim 25, wherein an automated expression classification system generates a set of predicted emotion probabilities for each respondent as statistical inferences.

27. The method according to claim 25, wherein an automated facial expression classification system generates a set of predicted emotion probabilities for each respondent as statistical inferences.

28. A system for assessing the impact of non-verbal responses of a respondent to a stimulus, said system comprising
a data processing unit with a reference stimulus, a stimulus under test and questions for said stimulus to be presented to the respondent;
an image device for recording an immediate non-verbal response of said respondent to said presented stimulus;
means for transmitting the recorded image of said non-verbal response across a communications network to an image processing unit;
said image processing unit comprising means for automatically calculating emotional probabilities of the non-verbal response(s) of the respondent from said images employing statistical techniques; and
said data processing unit comprises means for determining and emotion intensity score from said predicted classification probabilities and means for reporting at said a data processing unit an analysis of verbal response(s) by determined emotion intensity score.

29. A system according to claim 28, wherein said data processing unit and said image processing unit are located the same server.

30. A system according to claim 28, wherein said means for calculating an emotional intensity score.
